(19) United States
(12) Patent Application Publication
(10) Pub. No.: US 2009/0074848 A1
(43) Pub. Date: Mar. 19, 2009

(54) COMBINATION FORMULATIONS OF CYTIDINE ANALOGS AND PLATINUM AGENTS

(76) Inventors: Sharon Johnstone, Vancouver (CA); Pierrot Harvie, Vancouver (CA); Paul Tardi, Surrey (CA); Troy Harasym, North Vancouver (CA); Lawrence Mayer, North Vancouver (CA)

Correspondence Address: MORRISON & FOERSTER LLP, 12531 HIGH BLUFF DRIVE, SUITE 100, SAN DIEGO, CA, 92130-2040, US

(21) Appl. No.: 12/064,326

(22) PCT Filed: Sep. 19, 2006
(86) PCT No.: PCT/US06/36569

§ 371 Date: Aug. 6, 2008

Related U.S. Application Data
(60) Provisional application No. 60/718,505, filed on Sep. 19, 2005.

(51) Int. Cl. A61K 9/127; A61K 9/14; A61K 33/24; A61P 43/00; A61K 31/282; A61K 31/513

(52) U.S. Cl. 424/450; 424/489; 424/649; 514/492; 514/274

(57) ABSTRACT

Compositions which comprise delivery vehicles having stably associated therewith a cytidine analog and a platinum agent are useful in achieving enhanced therapeutic effects when combinations of these drugs are administered.

### CROSS REFERENCE TO RELATED APPLICATION

This application claims benefit of provisional application U.S. Ser. No. 60/718,505, filed 19 Sep. 2005. The contents of this application are incorporated herein by reference.

### TECHNICAL FIELD

The invention relates to compositions and methods for improved delivery of combinations of therapeutic agents. More particularly, the invention concerns delivery systems which provide combinations of cytidine analogs and platinum-based agents and derivatives thereof.

### BACKGROUND ART

The progression of many life-threatening diseases such as cancer, AIDS, infectious diseases, immune disorders and cardiovascular disorders are influenced by multiple molecular mechanisms. Due to this complexity, achieving cures with a single agent has been met with limited success. Thus, combinations of agents have often been used to combat disease, particularly in the treatment of cancers. It appears that there is a strong correlation between the number of agents administered and cure rates for cancers such as acute lymphocytic leukemia and metastatic colorectal cancer (Frei, et al., Clin. Cancer Res. (1998) 4:2027-2037; Fisher, M. D.; Clin Colorectal Cancer (2001) August; 1(2):85-6). In particular, combinations of cytidine analogs and platinum-based compounds are being investigated for their effects on many cancers including, non-small-cell lung, bladder, breast and ovarian cancers. Phase III clinical trials utilizing the platinum agents, cisplatin or carboplatin, plus the cytidine analog, gemcitabine, have demonstrated improved activity in patients with advanced non-small-cell lung cancers (Harper, P., Semin Oncology (2003) August; 30(4 Suppl 10):2-12). More recent clinical trials with gemcitabine and cisplatin in combination showed activity against advanced bladder cancers (Rosenberg, et al., Journal of Urology (2005) July; 174(1):14-20). This combination was shown to be as effective with less side effects as an older regimen known as MVAC (methotrexate, vinblastine, doxorubicin and cisplatin) and thus resulted in widespread use of gemcitabine/cisplatin as first line chemotherapy for metastatic bladder cancer.

Platinum-based drugs, such as cisplatin, carboplatin and oxaliplatin, are primarily employed because the platinum atom allows them to form DNA adducts which inhibit DNA synthesis and often induce programmed cell death (apoptosis). They exhibit activity against a wide range of tumors and are commonly used in combination therapies for solid tumors such as ovarian, lung, testicular, bladder, colorectal, gastric, head and neck and melanoma cancers. Specific platinum-based drugs are known to bind DNA in unique arrangements which may explain the tumor-specific activity demonstrated with different platinum agents. This has proven beneficial for second-line treatments against cancers that have exhibited resistance against a first-line treatment with a different platinum agent.

Cytidine analogs, examples of such analogs include: cytarabine, 5-Azacytidine, and gemcitabine, are known antineoplastic agents. For example, these compounds have demonstrated effectiveness at inhibiting DNA synthesis and repair in leukemia and cancer cells. These properties have enabled these compounds to effectively treat acute myelocytic leukemia, acute lymphoblastic leukemia and myelodysplastic syndromes, pancreatic cancer and lung cancer.

U.S. Pat. No. 5,464,826 discusses a method to treat a diseased patient by administering gemcitabine; however, no combinations with platinum-based drugs or pharmaceutical preparations designed to control drug delivery were suggested.

Similarly, U.S. 2004/0052864 discusses the administration of a nonencapsulated DNA methylation inhibitor and a nonencapsulated antineoplastic agent, either singularly or in a free drug cocktail, for the treatment of diseases associated with abnormal cell proliferation. However, no pharmaceutical preparations designed to control delivery or half-lives of the drugs were suggested in this publication.

There are various drawbacks that limit the therapeutic use of non-encapsulated drug cocktails. For instance, administration of free drug cocktails often results in rapid clearance of one or all of the drugs before reaching the tumor site. For this reason, many drugs have been incorporated into delivery vehicles designed to ‘shield’ them from mechanisms that would otherwise result in their clearance from the bloodstream. It is well known that liposomes have the ability to provide this ‘shielding’ effect and they are thus able to extend the half-life of therapeutic agents. However, formulation of specific drugs or more than one drug into delivery vehicles has proven to be difficult because the lipid composition of the vehicle often differentially affects the pharmacokinetics of individual drugs. Thus a composition that is suitable for retention and release of one drug may not be suitable for the retention and release of a second drug. Presently, although there are a number of active cytidine analog/platinum agent drug combinations being successfully utilized in clinical trials, a pharmaceutical preparation designed to control the pharmacokinetics, and thus tumor delivery, of both drugs has not been described.

For example, Zhang and Ahmad's PCT patent application WO 2004/017944 A1, claims a method for treating a cellular proliferative disease (such as cancer) by administering a combination of liposomes with a negative phospholipid and encapsulated gemcitabine with a free therapeutic agent other than gemcitabine. The application also claims a liposomal composition comprising gemcitabine and the negatively charged lipid, cardiolipin, which may further comprise one or more therapeutic agents which may be, for example, cisplatin or oxaliplatin; however, no pharmaceutical preparations designed to encapsulate and/or enhance circulation lifetimes of both drugs in the absence of cardiolipin were suggested.

Investigators of the present invention have identified particular delivery vehicle formulations required to accommodate a combination of a cytidine analog and a platinum agent (including gemcitabine and cisplatin), which result in superior drug loading and retention and sustained drug release of each agent. They have further demonstrated that synergistic ratios of these drugs, when encapsulated in liposomes, can be successfully maintained in the blood compartment over time and therefore lead to enhanced efficacy.

### DISCLOSURE OF THE INVENTION

The invention relates to compositions and methods for administering effective amounts of cytidine analog and platinum agent (e.g., cytarabine, 5-Azacytidine or gemcitabine with cisplatin, carboplatin or oxaliplatin) drug combinations using delivery vehicles that are stably associated therewith at least one cytidine analog and one platinum-based drug. These compositions allow the two or more agents to be delivered to the disease site in a coordinated fashion, thereby assuring that the agents will be present at the disease site at a desired ratio. This result will be achieved whether the agents are co-encapsulated in particulate delivery vehicles, or are encapsulated in separate delivery vehicles administered such that desired ratios are maintained at the disease site. The pharmacokinetics (PK) of the composition are controlled by the delivery vehicles themselves such that coordinated delivery is achieved (provided that the PK of the delivery systems are comparable).

Thus, in one aspect, the invention provides a composition for parenteral administration comprising at least one cytidine analog and one platinum agent associated with particulate delivery vehicles at therapeutically effective ratios, i.e. those that are non-antagonistic.

The therapeutically effective non-antagonistic ratio of the agents can be determined by a number of methods including:

i) in vitro assessment of the biological activity or effects of the agents on relevant cell culture or cell-free systems, as well as tumor homogenates from individual patient biopsies, over a range of concentrations; and,

ii) various in vivo assessments of activity based on comparisons of the combination with individual drugs at specific doses or with drugs at the Maximum Tolerated Dose (MTD).

Frequent combinations are gemcitabine with cisplatin or carboplatin, among other platinum-based drugs together with cytarabine, gemcitabine or other cytidine analogs. Any method which results in determination of a ratio of agents which maintains a desired therapeutic effect may be used.

The composition comprises at least one cytidine analog and one platinum agent in a mole ratio of the cytidine analog to the platinum agent which exhibits a desired biologic effect, where the ratio is that at which the agents are non-antagonistic. The ratio may be determined to be non-antagonistic in vitro by testing relevant cells in culture, cell-free systems or tumor homogenates. By “relevant” cells, applicants refer to at least one cell culture or cell line which is appropriate for testing the desired biological effect. As these agents are used as antineoplastic agents, “relevant” cells are those of cell lines identified by the Developmental Therapeutics Program (DTP) of the National Cancer Institute (NCI)/National Institutes of Health (NIH) as useful in their anticancer drug discovery program. Currently the DTP screen utilizes 60 different human tumor cell lines. The desired activity on at least one of such cell lines would need to be demonstrated. By “tumor homogenate,” the applicant refers to cells generated from the homogenization of patient biopsies or tumors. Extraction of whole tumors or tumor biopsies can be achieved through standard medical techniques by a qualified physician and homogenization of the tissue into single cells can be carried out in the laboratory using a number of methods well-known in the art. Alternatively, the ratio may be determined to be non-antagonistic in vivo based on efficacy studies. In these studies, the anti-tumor activity of individual agents encapsulated in the delivery vehicles is determined. These efficacy results are then compared to the drug combination in a delivery vehicles where the drug doses are the same as those used for the respective individual agents.

In another aspect, the invention is directed to a method to deliver a therapeutically effective amount of a cytidine analog:platinum agent combination (e.g., gemcitabine:cisplatin) to a desired target by administering the compositions of the invention.

The invention is also directed to a method to deliver a therapeutically effective amount of a cytidine analog:platinum agent combination by administering a cytidine analog stably associated with a first delivery vehicle and a platinum agent stably associated with a second delivery vehicle. The first and second delivery vehicles may be contained in separate vials, the contents of the vials being administered to a patient essentially simultaneously. In one embodiment, the ratio of the cytidine analog and the platinum agent is non-antagonistic.

In another aspect, the invention is directed to a method to prepare a therapeutic composition comprising delivery vehicles containing at least one cytidine analog and one platinum agent which provides a desired therapeutic effect, which method comprises providing a panel of at least one cytidine analog and one platinum agent wherein the panel comprises at least one, but preferably a multiplicity of ratios of said drugs, testing the ability of the members of the panel to exert a biological effect on a relevant cell culture or tumor homogenate over a range of concentrations, selecting a member of the panel wherein the ratio provides a desired therapeutic effect on said cell culture or tumor homogenate over a suitable range of concentrations; and stably associating the ratio of drugs into lipid-based drug delivery vehicles. In preferred embodiments, the above mentioned desired therapeutic effect is non-antagonistic.

As further described below, in one preferred embodiment, in designing an appropriate combination in accordance with the method described above, the non-antagonistic ratios are selected as those that have a combination index (CI) of 1.1 in vitro and/or show synergy in vivo. In further embodiments, suitable formulations are designed such that they stably incorporate an effective amount of a cytidine analog:platinum agent combination (e.g., gemcitabine:cisplatin) and allow for the sustained release of both drugs in vivo. Preferred liposomal formulations contain at least one negatively charged lipid, such as phosphatidylglycerol.

### BRIEF DESCRIPTION OF THE DRAWINGS

FIG. 1A is a graph showing the combination index (CI) plotted as a function of the fraction of BxPc-3 human pancreatic carcinoma cells affected (fₐ) by combinations of gemcitabine:cisplatin at mole ratios ranging from 64:1 to 1:2048 (refer to FIG. 1A legend).

FIG. 1B is a graph showing the combination (CI) plotted as a function of the fraction of H460 human non-small cell lung carcinoma cells affected (fₐ) by combinations of gemcitabine:cisplatin at mole ratios ranging from 64:1 to 1:2048 (refer to FIG. 1B legend).

FIG. 2A is a graph of the plasma drug concentrations of gemcitabine and cisplatin as a function of time after intravenous administration to CD-1 mice of co-encapsulated gemcitabine and cisplatin in DSPC:DSPG:CHOL (7:2:1 mol ratio) at a gemcitabine:cisplatin molar ratio of about 1:1.

FIG. 2B is a graph of the gemcitabine:cisplatin ratio in the plasma as a function of time after intravenous administration of co-encapsulated gemcitabine and cisplatin in DSPC:DSPG:CHOL (7:2:1 mol ratio) liposomes at about a 1:1 molar ratio. Data points represent the molar ratios of gemcitabine:cisplatin determined in plasma (+/−standard deviation) at the specified time points.

FIG. 2C is a graph of the gemcitabine:cisplatin ratio in the plasma as a function of time after intravenous administration of co-encapsulated gemcitabine and cisplatin in DSPC:DSPG:CHOL (7:2:1 mol ratio) liposomes at about a 3:1 molar ratio. Data points represent the molar ratios of gemcitabine:cisplatin determined in plasma (+/−standard deviation) at the specified time points.

FIG. 2D is a graph of the gemcitabine:cisplatin ratio in the plasma as a function of time after intravenous administration of co-encapsulated gemcitabine and cisplatin in DSPC:DSPG:CHOL (7:2:1 mol ratio) liposomes at about a 6:1 gemcitabine:cisplatin molar ratio. Data points represent the molar ratios of gemcitabine:cisplatin determined in plasma (+/−standard deviation) at the specified time points.

FIG. 2E is a graph of the gemcitabine:cisplatin ratio in the plasma as a function of time after intravenous administration of co-encapsulated gemcitabine and cisplatin in DSPC:DSPG:CHOL (7:2:1 mol ratio) liposomes at about a 10:1 molar ratio. Data points represent the molar ratios of gemcitabine:cisplatin determined in plasma (+/−standard deviation) at the specified time points.

FIG. 3A is a graph of the plasma drug concentrations of gemcitabine and cisplatin as a function of time after intravenous administration to B6D2F1/Hsd mice of separately loaded DSPC:DSPG:CHOL (7:2:1 mol ratio) liposomes at a targeted gemcitabine:cisplatin molar ratio of 3:1.

FIG. 3B is a graph of the gemcitabine:cisplatin ratio (mol:mol) in the plasma as a function of time after intravenous administration to B6D2F1/Hsd mice of separately loaded DSPC:DSPG:CHOL (7:2:1 mol ratio) liposomes at a targeted gemcitabine:cisplatin molar ratio of 3:1.

FIG. 4A is a graph of the plasma drug concentrations of gemcitabine and cisplatin as a function of time after intravenous administration to B6D2F1/Hsd mice of separately loaded DSPC:DPPC:DSPG:CHOL (35:35:20:10) liposomes at a targeted gemcitabine:cisplatin molar ratio of 3:1. P FIG. 4B is a graph of the gemcitabine:cisplatin ratio in the plasma as a function of time after intravenous administration to B6D2F1/Hsd mice of separately loaded DSPC:DPPC:DSPG:CHOL (35:35:20:10) liposomes at a targeted gemcitabine:cisplatin molar ratio of 3:1.

FIG. 5 is a graph of the anti-cancer efficacy in the lymphocytic leukemia P388 ascites tumor model of separately loaded gemcitabine and cisplatin in the DSPC:DSPG:Chol (7:2:1 mol:mol) formulation. Mice were administered intravenously with saline control, 4.0 mg/kg liposomal gemcitabine, 1.0 mg/kg liposomal cisplatin, or a fixed 3:1 molar ratio of liposomal gemcitabine (4.0 mg/kg) and liposomal cisplatin (1.5 mg/kg).

FIG. 6 is a graph of the anti-cancer efficacy in the lymphocytic leukemia P388 ascites tumor model of separately loaded DSPC:DPPC:DPPG:Chol (35:35:20:10) gemcitabine and cisplatin-containing liposomes. Mice were administered intravenously with saline control, 5.3 mg/kg liposomal gemcitabine, 2 mg/kg liposomal cisplatin, or 5.3 mg/kg liposomal gemcitabine and 2 mg/kg liposomal cisplatin. These doses represent approximately 50% of the maximum tolerable dose. Efficacy was evaluated as increased life span and log cell kill.

### MODES OF CARRYING OUT THE INVENTION¹

Unless defined otherwise, all terms of art, notations and other scientific terms or terminology used herein have the same meaning as is commonly understood by one of ordinary skill in the art to which this invention belongs. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference. ¹Abbreviations DSPC: distearoylphosphatidylcholine; PG: phosphatidylglycerol; DSPG: distearoylphosphatidylglycerol; PI: phosphatidylinositol; SM: sphingomyelin; Chol or CH: cholesterol; CHE: cholesteryl hexadecyl ether; SUV: small unilamellar vesicle; LUV: large unilamellar vesicle; MLV: multilamellar vesicle; MTT: 3-(4,5-dimethylthiazol-2-yl)-2,5-diphenyl tetrazolium bromide; EDTA: ethylenediaminetetraacetic acid; HEPES: N-[2-hydroxylethyl]-piperazine-N-[2-ethanesulfonic acid]; HBS: HEPES buffered saline (20 mM HEPES, 150 mM NaCl, pH 7.4); SHE: 300 mM sucrose, 20 mM HEPES, 30 mM EDTA; TEA: triethanolamine; CI: combination index; fₐ: fraction affected.

As used herein, “a” or “an” means “at least one” or “one or more.”

The invention provides compositions comprising delivery vehicles stably associated therewith at least one cytidine analog (e.g., gemcitabine) and one platinum agent (e.g., cisplatin), wherein the cytidine analog and platinum agent are present at cytidine analog:platinum agent (e.g., gemcitabine:cisplatin) mole ratios that exhibit a desired cytotoxic, cytostatic or biologic effect to relevant cells, tumor homogenates or animal tumor models.

As used herein, “stably associated” or “encapsulation” is meant to mean stable association with the delivery vehicle. Thus, it is not necessary for the vehicle to surround the agent or agents as long as the agent or agents is/are stably associated with the vehicles when administered in vivo. Thus, “stably associated with” and “encapsulated in” or “encapsulated with” or “co-encapsulated in or with” are intended to be synonymous terms. They are used interchangeably in this specification. The stable association may be effected by a variety of means, including covalent bonding to the delivery vehicle, preferably with a cleavable linkage, noncovalent bonding, and trapping the agent in the interior of the delivery vehicle and the like. The association must be sufficiently stable so that the agents remain associated with the delivery vehicle at a non-antagonistic ratio until it is delivered to the target site in the treated subject. This can be assessed by measuring the relative concentration of the agents in blood or plasma as a function of time to assure that a non-antagonistic ratio is maintained for a sufficient time to allow delivery to the target.

The delivery vehicles useful in the invention comprise delivery vehicles suitable for parenteral administration. Such delivery vehicles include liposomes, lipid micelles, polymer-based vehicles, nanoparticles, and the like. Any particulate delivery vehicle which controls pharmacokinetics and release of the stably associated agents at the target may be used.

In one embodiment, the compositions will include liposomes stably associated therewith at least one cytidine analog and one platinum agent in a mole ratio of the cytidine analog:platinum agent which exhibits a non-antagonistic effect. In one aspect of the invention, liposomes which comprise phosphatidylcholine are provided, preferably distearoylphosphatidylcholine. In another aspect of the invention, liposomes which comprise a sterol are provided. Preferably the sterol is cholesterol.

In further embodiments of the invention, the delivery vehicles comprise a third or fourth agent. Any therapeutic, diagnostic or cosmetic agent may be included.

The delivery vehicles of the present invention may be used in parenteral administration as well as inclusion in an implantable device at or near the target site for therapeutic purposes or medical imaging and the like. Preferably, the delivery vehicles of the invention are used in parenteral administration, most preferably, intravenous administration.

The preferred embodiments herein described are not intended to be exhaustive or to limit the scope of the invention to the precise forms disclosed. They are chosen and described to best explain the principles of the invention and its application and practical use to allow others skilled in the art to comprehend its teachings.

Cytidine Analogs

Antimetabolites or, more particularly, cytidine analogs such as cytarabine, 5-Azacytidine, and gemcitabine (2′,2′-Difluorodeoxycytidine) are known antineoplastic agents. Cytidine analogs may also be referred to in the art as cytosine nucleoside analogs. Antimetabolites are compounds that are similar enough to a natural chemical to participate in a normal biochemical reaction in cells but different enough to interfere with the normal division and functions of cells. These compounds generally inhibit a normal metabolic process. Cytarabine is a pyrimidine nucleoside antimetabolite. This compound is an analog of 2′-deoxycytidine with the 2′-hydroxyl in a position trans to the 3′-hydroxyl of the sugar. Cytarabine is considered equivalent with 4-Amino-1-β-d-arabinofuranosyl-2(1H)-pyrimidinone, 1-β-d-arabinofuranosylcytosine, Ara-C, β-cytosine arabinoside, aracytidine, CHX-3311, U-19920, Alexan, Arabitin, Aracytine, Cytarbel, Cytosar, Erpalfa, Iretan and Udicil. In cytidine analogs such as cytarabine, the sugar moiety comprises an arabinose rather than ribose. Cytarabine is recognized as useful in the therapy of acute myelocytic leukemia (AML) and has proven effectiveness in the remission of this disorder. However, the mechanism of action of cytarabine is uncertain, nevertheless incorporation of this nucleotide into DNA leads to an inhibition of polymerization by termination of strand synthesis.

Cytarabine must be “activated” via conversion of the 5-monophosphate nucleotide (AraCMP) to terminate strand synthesis. AraCMP is then able to react with selected nucleotide kinases to form diphosphate and triphosphate nucleotides (AraCDP and AraCTP). Cytarabine incorporation into DNA is S-phase specific, thus dosing has been advocated over at least one full cell cycle to obtain inhibition of DNA synthesis. Inhibition of DNA synthesis occurs at low AraCTP concentrations and inhibits DNA chain elongation by incorporation of AraC into the terminal portion of a growing DNA chain. Moreover, there appears to be a correlation between the amount of AraC incorporated into the chain and the inhibition of DNA synthesis.

Subjects can develop resistance to cytarabine. Such resistance is generally due to a deficiency of deoxycytidine kinase, which produces AraCMP. In addition, degrative enzymes such as cytidine deaminase (which deaminates AraC to nontoxic arauridine) and dCMP (which converts AraCMP to inactive AraUMP) also affect efficacy.

5-Azacytidine (Azacytidine; 5-AzaC) is a compound that exhibits antineoplastic activity. This compound is known as useful for the treatment of AML, acute lymphoblastic leukemia and myelodysplastic syndromes. Current studies are evaluating the effects of this compound in beta thalassemia, acute myeloid leukemia, myelodysplastic syndrome, advanced or metastatic solid tumors, non-Hodgkin's lymphoma, multiple myeloma, non-small cell lung cancer and prostate cancer. 5-AzaC has been shown to inhibit DNA methylation, which in turn affects gene expression. Side effects include decreased white and red blood cell and platelet count, nausea, vomiting, fatigue, diarrhea, among other effects.

Gemcitabine is a nucleoside analog that exhibits antitumor activity. Gemcitabine HCl consists of a 2′-deoxy-2′,2′-difluorocytidine monohydrochloride (β-isomer) and is known as effective in treating pancreatic, bladder, breast and lung cancers. In general, gemcitabine prevents cells from making DNA and RNA by interfering with the synthesis of nucleic acids. This action stops the growth of cancer cells, causing the cells to die. Side effects include decreased white blood cell and platelet count, nausea, vomiting, fatigue, diarrhea, flu-like symptoms, rashes, among other effects.

Platinum Agents

In recent years metal-based therapeutic agents have played a relevant role in chemotherapy treatments. In particular, platinum-based compounds, are proving to be some of the most effective anticancer drugs used in clinical practice. Platinum agents, as incorporated herein, are used primarily because they form DNA adducts that block DNA and RNA synthesis and apoptosis. The nature of the platinum/DNA adducts has been widely investigated by hydrolysis of DNA into nucleotides. Studies have shown that the adduct is typically a cross link involving the N-7 of DNA purine (adenine (A) and guanine (G)) bases. The preferential complex for platinum compounds such as cisplatin, is an intrastrand crosslink at the dinucleotides GG (62% occurrence) and AG (22% occurrence). In fact, intrastrand cross linking has been shown to correlate with the clinical response to cisplatin therapy. To possess this type of antitumor activity a platinum agent must have two relatively labile leaving groups to react with the DNA bases. Typically, platinum agents have a central platinum atom bonded to four ligands, two of which are reactive. In the case of cisplatin, the platinum atom is linked to two amino groups and two chloride (leaving) groups.

“Platinum agents,” therefore refers to therapeutic drugs that contain a reactive platinum atom, including derivatized and underivatized forms of cisplatin and its related compounds which have the essential features of containing a reactive platinum atom.

Approximately 3,000 platinum analogs have been synthesized over the past 30 years; however, only 6 are presently in clinical development, including cisplatin, carboplatin and oxaliplatin. Cisplatin and carboplatin dominate the world platinum/cancer market and have become critical elements in the standard practice of care for numerous solid tumors including, ovarian, lung, testicular, bladder, gastric, melanoma and head and neck cancers. Oxaliplatin (a newer platinum) has a different mechanism of action than either cisplatin or carboplatin which has proven to be especially important in cisplatin-resistant models and cell lines expressing resistance genes.

Cisplatin (cis-diamminedichoroplatinum (II)) has a well-established activity spectrum, mainly in the treatment of lung, ovarian, and germ cell tumors. Its binding to DNA has been shown to cause the DNA duplex to bend and unwind. This coordination to the DNA leads to not only inhibition of DNA replication and transcription, but also to apoptosis. Interestingly, the cytotoxic activity of cisplatin is dependent upon its stereochemistry, as the trans isomer (which binds DNA differently) exhibits reduced activity compared to the cis isomer.

Carboplatin (cis-diammine-1,1-cyclobutanedicarboxylateplatinum) is one of the best-characterized cisplatin analogs. It was approved by the United States Food and Drug Administration (FDA) in 1989 for treatment of human ovarian cancers. It is known to form an identical type of DNA adduct and exhibit the same therapeutic effects as cisplatin. However, carboplatin requires lower doses to achieve these same affects and it displays significantly less toxicity to the peripheral nervous system and kidneys. It is believed that this is likely due to the presence of a bidentate dicarboxylate ligand in carboplatin which slows down its degradation into potentially damaging derivatives.

Oxaliplatin (trans-1-diaminocyclohexane oxalatoplatinum) is a new platinum salt that belongs to the DACH (diaminocyclohexane) platinum family, and is the only such cisplatin analog that has entered clinical development and achieved approval for marketing. It demonstrates good clinical tolerance with the absence of renal or auditory toxicity. The exact mechanism of action of oxaliplatin is unclear. It is known to form reactive platinum complexes which are believed to inhibit DNA synthesis by forming interstrand and intrastrand cross-linking of DNA molecules; however, it binds in a different location on DNA than cisplatin or carboplatin. It is also a larger and bulkier compound than either cisplatin or carboplatin, which makes it harder to separate from the DNA once bound. Another difference between oxaliplatin and the other platinum compounds is its spectrum of cytotoxicity, or ability to induce cell death. In particular, it has shown activity against six colorectal cell lines that both cisplatin and carboplatin have limited cytotoxicity against, demonstrating the selective nature of these platinum-based compounds.

In one embodiment of the invention, the “platinum agent” is selected based on its activity against a particular cell type or tumor. In preferred embodiments, the platinum agent is cisplatin, carboplatin or oxaliplatin. Most preferably, the platinum agent is cisplatin or carboplatin.

The effects of combining cytidine analogs and platinum agents in liposomes of the invention will act to inhibit DNA synthesis through multiple pathways and likely induce apoptosis. This is critical for the treatment of hyperproliferative diseases such as cancer, and importantly, may prove superior for treating multiple-drug resistant cancers such as cisplatin-resistant tumors.

Determining Non-Antagonistic Gemcitabine:Cisplatin Ratios

Cytidine analogs and platinum agents will be encapsulated into delivery vehicles at synergistic or additive (i.e., non-antagonistic) ratios. Determination of ratios of agents that display synergistic or additive combination effects may be carried out in vitro using various algorithms, based on the types of experimental data described below. These methods include isobologram methods (Loewe, et al., Arzneim-Forsch (1953) 3:285-290; Steel, et al., Int. J. Radiol. Oncol. Biol. Phys. (1979) 5:27-55), the fractional product method (Webb, Enzyme and Metabolic Inhibitors (1963) Vol. 1, pp. 1-5. New York: Academic Press), CombiTool, ComboStat and the Chou-Talalay median-effect method based on an equation described in Chou (J. Theor. Biol. (1976) 39:253-276; and Chou, Mol. Pharmacol. (1974) 10:235-247). Alternatives include surviving fraction (Zoli, et al., Int. J. Cancer (1999) 80:413-416), percentage response to granulocyte/macrophage-colony forming unit compared with controls (Pannacciulli, et al., Anticancer Res. (1999) 19:409-412) and others (Berenbaum, Pharmacol. Rev. (1989) 41:93-141; Greco, et al., Pharmacol Rev. (1995) 47:331-385).

The Chou-Talalay median-effect method is preferred for in vitro analysis. The analysis utilizes an equation wherein the dose that causes a particular effect, fₐ, is given by:

D=Dₘ[fₐ/(1−fₐ)]^{1/m}

in which D is the dose of the drug used, fₐ is the fraction of cells affected by that dose, Dₘ is the dose for median effect signifying the potency and m is a coefficient representing the shape of the dose-effect curve (m is 1 for first order reactions).

The data obtained from the latter equation is used in a second equation, the combination index equation, to obtain combination index values (CI), which are used to indicate the extent of synergism, antagonism, or additivity. The combination index equation is based on the multiple drug-effect equation of Chou-Talalay derived from enzyme kinetic models as described by Chou and Talalay, Adv. Enzyme Reg. (1984) 22:27-55; and by Chou, et al., in: Synergism and Antagonism in Chemotherapy, Chou and Rideout, eds., Academic Press: New York 1991:223-244. A computer program (CalcuSyn) for this calculation is found in Chou and Chou (“Dose-effect analysis with microcomputers: quantitation of ED50, LD50, synergism, antagonism, low-dose risk, receptor ligand binding and enzyme kinetics”: CalcuSyn Manual and Software; Cambridge: Biosoft 1987).

According to the CalcuSyn program, synergism is defined as a more than expected additive effect, and antagonism as a less than expected additive effect. Chou and Talalay in 1983 proposed the designation of CI=1 as the additive effect, thus from the multiple drug effect equation of two drugs, we obtain:

CI=(D)₁/(Dₓ)₁+(D)₂/(Dₓ)₂ [Eq. 1]

for mutually exclusive drugs that have the same or similar modes of action, and it is further proposed that

CI=(D)₁/(Dₓ)₁+(D)₂/(Dₓ)₂+((D)₁(D)₂)/(Dₓ)₁(Dₓ)₂ [Eq. 2]

for mutually non-exclusive drugs that have totally independent modes of action. CI<1, =1, and >1 indicates synergism, additive effect, and antagonism, respectively. Equation 1 or equation 2 dictates that drug 1, (D)₁, and drug 2, (D)₂, (in the numerators) in combination inhibit x % in the actual experiment. Thus, the experimentally observed x % inhibition may not be a round number but most frequently has a decimal fraction. (Dₓ)₁ and (Dₓ)₂ (in the denominators) of equations 1 and 2 are the doses of drug 1 and drug 2 alone, respectively, inhibiting x %.

For simplicity, mutual exclusivity is usually assumed when more than two drugs are involved in combinations (CalcuSyn Manual and Software; Cambridge: Biosoft 1987).

A two-drug combination may be further used as a single pharmaceutical unit to determine synergistic or additive interactions with a third agent. In addition, a three-agent combination may be used as a unit to determine non-antagonistic interactions with a fourth agent, and so on.

As set forth above, the in vitro studies on cell cultures will be conducted with “relevant” cells. The choice of cells will depend on the intended therapeutic use of the agent. Only one relevant cell line or cell culture type need exhibit the required non-antagonistic effect in order to provide a basis for the compositions to come within the scope of the invention.

For example, in one preferred embodiment of the invention, the combination of agents is intended for anticancer therapy. In a frequent embodiment, the combination of agents is intended for leukemia or lymphoma therapy. Appropriate choices will then be made of the cells to be tested and the nature of the test. In particular, tumor cell lines are suitable subjects and measurement of cell death or cell stasis is an appropriate end point. As will be discussed further below, in the context of attempting to find suitable non-antagonistic combinations for other indications, other target cells and criteria other than cytotoxicity or cell stasis could be employed.

For determinations involving antitumor agents, cell lines may be obtained from standard cell line repositories (NCI or ATCC for example), from academic institutions or other organizations including commercial sources. Preferred cell lines would include one or more selected from cell lines identified by the Developmental Therapeutics Program of the NCI/NIH. The tumor cell line screen used by this program currently identifies 60 different tumor cell lines representing leukemia, melanoma, and cancers of the lung, colon, brain, ovary, breast, prostate and kidney. The required non-antagonistic effect over a desired concentration range need be shown only on a single cell type; however, it is preferred that at least two cell lines exhibit this effect, more preferably three cell lines, more preferably five cell lines, and more preferably 10 cell lines. The cell lines may be established tumor cell lines or primary cultures obtained from patient samples. The cell lines may be from any species but the preferred source will be mammalian and in particular human. The cell lines may be genetically altered by selection under various laboratory conditions, and/or by the addition or deletion of exogenous genetic material. Cell lines may be transfected by any gene-transfer technique, including but not limited to, viral or plasmid-based transfection methods. The modifications may include the transfer of cDNA encoding the expression of a specific protein or peptide, a regulatory element such as a promoter or enhancer sequence or antisense DNA or RNA. Genetically engineered tissue culture cell lines may include lines with and without tumor suppressor genes, that is, genes such as p53, pTEN and p16; and lines created through the use of dominant negative methods, gene insertion methods and other selection methods. Preferred tissue culture cell lines that may be used to quantify cell viability, e.g., to test antitumor agents, include, but are not limited to, P388, L1210, HL-60, MOLT-4, 14BM-3, WeHi-3, H460, MCF-7, SF-268, HT29, UCT-116, LS180, B16-F10, A549, Capan-1, CAOV-3, IGROV1, BXPC-3, MX-1 and MDA-MB-231.

In one preferred embodiment, the given effect (fₐ) refers to cell death or cell stasis after application of a cytotoxic agent to a cell culture. Cell death or viability may be measured, for example, using the following methods:

| CYTOTOXICITY ASSAY | REFERENCE |
|---|---|
| MTT assay | Mosmann, J. Immunol. Methods |
| | (1983) 65(1-2): 55-63. |
| Trypan blue dye exclusion | Bhuyan, et al., Experimental Cell |
| | Research (1976) 97: 275-280. |
| Radioactive tritium (³H)- | Senik, et al., Int. J. Cancer |
| thymidine incorporation or DNA | (1975) 16(6): 946-959. |
| intercalating assay | |
| Radioactive chromium-51 | Brunner, et al., Immunology |
| release assay | (1968) 14: 181-196. |
| Glutamate pyruvate transaminase, | Mitchell, et al., J. of Tissue Culture |
| creatine phosphokinase and lactate | Methods (1980) 6(3&4): 113-116. |
| dehydrogenase enzyme leakage | |
| Neutral red uptake | Borenfreund and Puerner, Toxicol. |
| | Lett. (1985) 39: 119-124. |
| Alkaline phosphatase activity | Kyle, et al., J. Toxicol. Environ. |
| | Health (1983) 12: 99-117. |
| Propidium iodide staining | Nieminen, et al., Toxicol. Appl. |
| | Pharmacol. (1992) 115: 147-155. |
| Bis-carboxyethyl- | Kolber, et al., J. Immunol. Methods |
| carboxyfluorescein | (1988) 108: 255-264. |
| (BCECF) retention | |
| Mitochondrial membrane | Johnson, et al., Proc. Natl. Acad. Sci. |
| potential | USA (1980) 77: 990-994. |
| Clonogenic Assays | Puck, et al., J. of Experimental |
| | Medicine (1956) 103: 273-283. |
| LIVE/DEAD Viability/ | Morris, Biotechniques (1990) 8: |
| Cytotoxicity assay | 296-308. |
| Sulforhodamine B (SRB) assays | Rubinstein, et al., J. Natl. Cancer |
| | Instit. (1990) 82: 1113-1118. |

Non-antagonistic ratios of two or more agents can be determined for disease indications other than cancer and this information can be used to prepare therapeutic formulations of two or more drugs for the treatment of these diseases. With respect to in vitro assays, many measurable endpoints can be selected from which to define drug synergy, provided those endpoints are therapeutically relevant for the specific disease.

As set forth above, the in vitro studies on cell cultures will be conducted with “relevant” cells. The choice of cells will depend on the intended therapeutic use of the agent. In vitro studies on individual patient biopsies or whole tumors can be conducted with “tumor homogenate,” generated from homogenization of the tumor sample(s) into single cells.

In one preferred embodiment, the given effect (fₐ) refers to cell death or cell stasis after application of a cytotoxic agent to a “relevant” cell culture or “tumor homogenate” (see Example 1). Cell death or viability may be measured using a number of methods known in the art.

In vivo determination of non-antagonistic ratios may be determined in a number of ways, including but not limited to, analysis of tumor growth inhibition, tumor regression, animal survival, or other end points of efficacy, such as biological markers of tumor growth, biochemical activity and/or expression of relevant target proteins in the cancer cells. The extent of anti-tumor activity as quantified using one of these efficacy endpoints is determined for the combination formulated at a specified ratio in a specified drug delivery vehicle. The amount of anti-tumor activity for the combination is compared with the amount of anti-tumor activity provided by the drugs administered individually in the same delivery vehicle as was used in the combination and at the same dose and schedule used for the combination. Non-antagonism is reflected by the combination in a delivery vehicle exhibiting increased anti-tumor activity over the individual delivery vehicle formulated agents such that it reflects additive or more than additive contributions of the two agents. Preferably, the log tumor cell kill provided by the treatments is used to quantify and compare the amount of anti-tumor activity. The log-cell kill is calculated based on the formula log₁₀ cell kill=tumor growth delay (in days)/(3.32×T_{d}) where T_{d} equals the tumor doubling time and where tumor growth delay is the treatment-induced increase in survival time for non-palpable tumor models or delay to reach a specific tumor size (e.g., 500 mg or 1 g) for solid tumor models in which the tumors can be measured directly using a caliper or other measuring device. Non-antagonistic activity of the combination would be indicated if the log cell kill of the combination is equal to or greater than the sum of the log cell kill values for the individual agents administered in the same delivery vehicle used for the combination and wherein the doses of the individual agents are the same as those used in the combination.

Preparation of Delivery Vehicles

When the appropriate ratios of the agents have been determined as described above, the agents at the appropriate ratio are placed into a delivery vehicle composition wherein one or more delivery vehicles encapsulates two or more agents. Not all the delivery vehicles in the composition need be identical. The delivery vehicles in the compositions are particles of sizes that depend on their route of administration, which can be suspended in an aqueous or other solvent and are able to encapsulate the agents of the invention. Such vehicles include, for example, lipid carriers, liposomes, cyclodextrins, polymer nanoparticles and polymer microparticles, including nanocapsules and nanospheres, block copolymer micelles, lipid stabilized emulsions, derivatized single-chain polymers, polymer lipid hybrid systems, lipid micelles, lipoprotein micelles as mentioned previously. For intravenous administration, delivery vehicles are typically about 4-6,000 nm in diameter. Preferred diameters are about 5-500 nm in diameter, more preferably 5-200 nm in diameter. For inhalation, intra-thecal, intra-articular, intra-arterial, intra-peritoneal or subcutaneous administration, delivery vehicles are typically from 4 μm to an excess of 50 μm. Delivery vehicle compositions designed for intra-ocular administration are generally smaller.

The therapeutic agents are “encapsulated” in the delivery vehicles. “Encapsulation,” as previously described, includes covalent or non-covalent association of an agent with the delivery vehicle. For example, this can be by interaction of the agent with the outer layer or layers of the delivery vehicle or entrapment of an agent within the delivery vehicle, equilibrium being achieved between different portions of the delivery vehicle. For example, for liposomes, encapsulation of an agent can be by association of the agent by interaction with the bilayer of the liposomes through covalent or non-covalent interaction with the lipid components or entrapment in the aqueous interior of the liposome, or in equilibrium between the internal aqueous phase and the bilayer. For polymer-based delivery vehicles, encapsulation can refer to covalent linkage of an agent to a linear or non-linear polymer. Further, non-limiting examples include the dispersion of agent throughout a polymer matrix, or the concentration of drug in the core of a nanocapsule, a block copolymer micelle or a polymer-lipid hybrid system. “Loading” refers to the act of encapsulating one or more agents into a delivery vehicle.

Encapsulation of the desired combination can be achieved either through encapsulation in separate delivery vehicles or within the same delivery vehicle. Where encapsulation into separate delivery vehicles, such as for example liposomes, is desired, the lipid composition of each liposome may be quite different to allow for coordinated pharmacokinetics. By altering the vehicle composition, release rates of encapsulated drugs can be matched to allow non-antagonistic ratios of the drugs to be delivered to the tumor site. Means of altering release rates include increasing the acyl-chain length of vesicle forming lipids to improve drug retention, controlling the exchange of surface grafted hydrophilic polymers such as PEG out of the liposome membrane and incorporating membrane-rigidifying agents such as sterols or sphingomyelin into the membrane. It should be apparent to those skilled in the art that if a first and second drug are desired to be administered at a specific drug ratio and if the second drug is retained poorly within the liposome composition of the first drug (e.g., DMPC/Chol), that improved pharmacokinetics may be achieved by encapsulating the second drug in a liposome composition with lipids of increased acyl chain length (e.g., DSPC/Chol). Alternatively, two or more agents may be encapsulated within the same delivery vehicle.

Techniques for encapsulation are dependent on the nature of the delivery vehicles.

As noted above, “encapsulated” simply means “stably associated” such that the delivery vehicles control the pharmacokinetics and maintain a non-antagonistic ratio of the agents until the agents are delivered to the target. Typically, stable association is measured by the ability of the composition to maintain a non-antagonistic ratio close to that administered in the blood or plasma over a suitable length of time so that it is assured that the ratio is delivered to the target tissue. As there is exchange between the intracellular regions of the target tissue and the plasma, the ratio in plasma or blood is a measure of maintenance of the ratio as delivered.

In one embodiment, compositions in accordance with this invention are used to treat cancer. Delivery of encapsulated drugs to a tumor site is achieved by administration of compositions of the invention. It is preferred that the delivery vehicles have a diameter of less than 300 nm or less than 200 nm. Tumor vasculature is generally leakier than normal vasculature due to fenestrations or gaps in the endothelia. This allows delivery vehicles of nanometer dimensions to penetrate the discontinuous endothelial cell layer and underlying basement membrane surrounding the vessels supplying blood to a tumor. Selective accumulation of the delivery vehicles into tumor sites following extravasation leads to enhanced anticancer drug delivery and therapeutic effectiveness.

In one embodiment, carriers for use in this invention are liposomes. Liposomes can be prepared as described in Liposomes: Rational Design (A. S. Janoff, ed., Marcel Dekker, Inc., New York, N.Y.), or by additional techniques known to those knowledgeable in the art. Suitable liposomes for use in this invention include large unilamellar vesicles (LUVs), multilamellar vesicles (MLVs), small unilamellar vesicles (SUVs) and interdigitating fusion liposomes.

Liposomes for use in this invention may be prepared to contain a phosphatidylcholine lipid, such as distearylphosphatidylcholine. Liposomes of the invention may also contain a sterol, such as cholesterol. Liposomes may also contain therapeutic lipids, which examples include ether lipids, phosphatidic acid, phosphonates, ceramide and ceramide analogs, sphingosine and sphingosine analogs and serine-containing lipids.

Liposomes may also be prepared with surface stabilizing hydrophilic polymer-lipid conjugates such as polyethylene glycol-DSPE, to enhance circulation longevity. The incorporation of negatively charged lipids such as phosphatidylglycerol (PG) and phosphatidylinositol (PI) may also be added to liposome formulations to increase the circulation longevity of the carrier. These lipids may be employed to replace hydrophilic polymer-lipid conjugates as surface stabilizing agents. Preferred embodiments of this invention may make use of liposomes containing phosphatidylglycerol (PG) or phosphatidylinositol (PI) to prevent aggregation thereby increasing the blood residence time of the carrier.

Various methods may be utilized to encapsulate active agents in liposomes. “Encapsulation” includes covalent or non-covalent association of an agent with the lipid-based delivery vehicle. For example, this can be achieved by interaction of the agent with the outer layer or layers of the liposome or entrapment of an agent within the liposome, equilibrium being achieved between different portions of the liposome. Thus encapsulation of an agent can be by association of the agent by interaction with the bilayer of the liposomes through covalent or non-covalent interaction with the lipid components or entrapment in the aqueous interior of the liposome, or in equilibrium between the internal aqueous phase and the bilayer. “Loading” refers to the act of encapsulating one or more agents into a delivery vehicle.

Encapsulation of the desired combination can be achieved either through encapsulation in separate delivery vehicles or within the same delivery vehicle. Where encapsulation into separate liposomes is desired, the lipid composition of each liposome may be quite different to allow for coordinated pharmacokinetics. By altering the vehicle composition, release rates of encapsulated drugs can be matched to allow desired ratios of the drugs to be delivered to the tumor site. Means of altering release rates include increasing the acyl-chain length of vesicle forming lipids to improve drug retention, controlling the exchange of surface grafted hydrophilic polymers such as PEG out of the liposome membrane and incorporating membrane-rigidifying agents such as sterols or sphingomyelin into the membrane. It should be apparent to those skilled in the art that if a first and second drug are desired to be administered at a specific drug ratio, and if the second drug is retained poorly within the liposome composition of the first drug (e.g., DMPC/Chol), that improved pharmacokinetics may be achieved by encapsulating the second drug in a liposoine composition with lipids of increased acyl chain length (e.g., DSPC/Chol). When encapsulated in separate liposomes, it should be readily accepted that ratios of cytidine analogs-to-platinum agents that have been determined on a patient-specific basis to provide optimal therapeutic activity can be generated for individual patients by combining the appropriate amounts of each liposome-encapsulated drug prior to administration. Alternatively, two or more agents may be encapsulated within the same liposome.

Techniques for encapsulation are dependent on the nature of the delivery vehicles. For example, therapeutic agents may be loaded into liposomes using both passive and active loading methods. Passive methods of encapsulating active agents in liposomes involve encapsulating the agent during the preparation of the liposomes. This includes a passive entrapment method described by Bangham, et al. (J. Mol. Biol. (1965) 12:238). This technique results in the formation of multilamellar vesicles (MLVs) that can be converted to large unilamellar vesicles (LUVs) or small unilamellar vesicles (SUVs) upon extrusion. Another suitable method of passive encapsulation includes an ether injection technique described by Deamer and Bangham (Biochim. Biophys. Acta (1976) 443:629) and the Reverse Phase Evaporation technique as described by Szoka and Papahadjopoulos (P.N.A.S. (1978) 75:4194). In addition, another suitable method of passive encapsulation involves passive equilibration after the formation of liposomes. This process involves incubating pre-formed liposomes under altered or non-ambient (based on temperature, pressure, etc.) conditions and adding a therapeutic agent (e.g., cytidine analog or anthracycline agent) to the exterior of the liposomes. The therapeutic agent then equilibrates into the interior of the liposomes, across the liposomal membrane. The liposomes are then returned to ambient conditions and unencapsulated therapeutic agent, if present, is removed via dialysis or another suitable method.

Active methods of encapsulation include the pH gradient loading technique described in U.S. Pat. Nos. 5,616,341, 5,736,155 and 5,785,987 and active metal-loading. One method of pH gradient loading is the citrate-base loading method utilizing citrate as the internal buffer at a pH of 4.0 and a neutral exterior buffer. Other methods employed to establish and maintain a pH gradient across a liposome involve the use of an ionophore that can insert into the liposome membrane and transportions across membranes in exchange for protons (see U.S. Pat. No. 5,837,282). A recent and preferred technique utilizing transition metals to drive the uptake of drugs into liposomes via complexation in the absence of an ionophore may also be used (outlined in U.S. Publication 2003/0091621 and incorporated by reference herein). This technique relies on the formation of a drug-metal complex rather than the establishment of a pH gradient to drive uptake of drug.

Passive and active methods of entrapment may also be coupled in order to prepare a liposome formulation containing more than one encapsulated agent.

While an embodiment wherein the delivery vehicles or liposomes has been extensively discussed, it will be recognized by the skilled artisan that alternative delivery vehicles are also appropriate and methods for stable association with such alternatives are well known in the art. Thus, nanoparticulates, polymer-based particulates, micelles, and the like may also be used.

Administering Compositions of the Invention In Vivo

As mentioned above, the delivery vehicle compositions of the present invention may be administered to warm-blooded animals, including humans as well as to other animals such as mammals or avians. For treatment of human ailments, a qualified physician will determine how the compositions of the present invention should be utilized with respect to dose, schedule and route of administration using established protocols. Such applications may also utilize dose escalation should agents encapsulated in delivery vehicle compositions of the present invention exhibit reduced toxicity to healthy tissues of the subject.

Preferably, the pharmaceutical compositions of the present invention are administered parenterally, i.e., intraarterially, intravenously, intraperitoneally, subcutaneously, or intramuscularly. More preferably, the pharmaceutical compositions are administered intravenously or intraperitoneally by a bolus or infusional injection. For example, see Rahman, et al, U.S. Pat. No. 3,993,754; Sears, U.S. Pat. No. 4,145,410; Papahadjopoulos, et al., U.S. Pat. No. 4,235,871; Schneider, U.S. Pat. No. 4,224,179; Lenk, et al., U.S. Pat. No. 4,522,803; and Fountain, et al., U.S. Pat. No. 4,588,578, incorporated by reference.

In other methods, the pharmaceutical or cosmetic preparations of the present invention can be contacted with the target tissue by direct application of the preparation to the tissue. The application may be made by topical, “open” or “closed” procedures. By “topical”, it is meant the direct application of the multi-drug preparation to a tissue exposed to the environment, such as the skin, oropharynx, external auditory canal, and the like. “Open” procedures are those procedures that include incising the skin of a patient and directly visualizing the underlying tissue to which the pharmaceutical preparations are applied. This is generally accomplished by a surgical procedure, such as a thoracotomy to access the lungs, abdominal laparotomy to access abdominal viscera, or other direct surgical approach to the target tissue. “Closed” procedures are invasive procedures in which the internal target tissues are not directly visualized, but accessed via inserting instruments through small wounds in the skin. For example, the preparations may be administered to the peritoneum by needle lavage. Alternatively, the preparations may be administered through endoscopic devices.

Pharmaceutical compositions comprising delivery vehicles of the invention are prepared according to standard techniques and may comprise water, buffered water, 0.9% saline, 0.3% glycine, 5% dextrose, iso-osmotic sucrose solutions and the like, including glycoproteins for enhanced stability, such as albumin, lipoprotein, globulin, and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. Additionally, the delivery vehicle suspension may include lipid-protective agents which protect lipids against free-radical and lipid-peroxidative damages on storage. Lipophilic free-radical quenchers, such as alpha-tocopherol and water-soluble iron-specific chelators, such as ferrioxamine, are suitable.

The concentration of delivery vehicles in the pharmaceutical formulations can vary widely, such as from less than about 0.05%, usually at or at least about 2-5% to as much as 10 to 30% by weight and will be selected primarily by fluid volumes, viscosities, and the like, in accordance with the particular mode of administration selected. For example, the concentration may be increased to lower the fluid load associated with treatment. Alternatively, delivery vehicles composed of irritating lipids may be diluted to low concentrations to lessen inflammation at the site of administration. For diagnosis, the amount of delivery vehicles administered will depend upon the particular label used, the disease state being diagnosed and the judgment of the clinician.

Preferably, the pharmaceutical compositions of the present invention are administered intravenously. Dosage for the delivery vehicle formulations will depend on the ratio of drug to lipid and the administrating physician's opinion based on age, weight, and condition of the patient.

In addition to pharmaceutical compositions, suitable formulations for veterinary use may be prepared and administered in a manner suitable to the subject. Preferred veterinary subjects include, for example, non-human primates, dogs, cats, cattle, horses and sheep. Subjects may also include laboratory animals, for example, in particular, rats, rabbits, mice, and guinea pigs.

Kits

The therapeutic agents in the invention compositions may be formulated separately in individual compositions wherein each therapeutic agent is stably associated with appropriate delivery vehicles. These compositions can be administered separately to subjects as long as the pharmacokinetics of the delivery vehicles are coordinated so that the ratio of therapeutic agents administered is maintained at the target for treatment. Thus, it is useful to construct kits which include, in separate containers, a first composition comprising delivery vehicles stably associated with at least a first therapeutic agent and, in a second container, a second composition comprising delivery vehicles stably associated with at least one second therapeutic agent. The containers can then be packaged into the kit.

The kit will also include instructions as to the mode of administration of the compositions to a subject, at least including a description of the ratio of amounts of each composition to be administered. Alternatively, or in addition, the kit is constructed so that the amounts of compositions in each container is pre-measured so that the contents of one container in combination with the contents of the other represent the correct ratio. Alternatively, or in addition, the containers may be marked with a measuring scale permitting dispensation of appropriate amounts according to the scales visible. The containers may themselves be useable in administration; for example, the kit might contain the appropriate amounts of each composition in separate syringes. Formulations which comprise the pre-formulated correct ratio of therapeutic agents may also be packaged in this way so that the formulation is administered directly from a syringe prepackaged in the kit.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

### EXAMPLES

### Example 1

### Gemcitabine:Cisplatin Synergy In Vitro is Drug Ratio Dependent

Many combinations of two or more drugs have the ability to exhibit synergistic effects. Similarly, combinations of the same two or more drugs may also show additive or antagonistic interactions. In order to identify ratios of gemcitabine and cisplatin that are synergistic, various ratios of gemcitabine and cisplatin were tested for their cytotoxic effects in vitro.

Measuring additive, synergistic or antagonistic effects was performed using gemcitabine:cisplatin at 18 ratios ranging from 64:1 to 1:2048 in BxPc-3 human pancreatic carcinoma and H460 human non-small-cell lung carcinoma cell lines. The standard tetrazolium-based colorimetric MTT cytotoxicity assay protocol (Mosmann, et al., J. Immunol. Methods (1983) 65(1-2):55-63) was utilized to determine the readout for the fraction of cells affected. Briefly, viable cells reduce the tetrazolium salt, 3-(4,5-diethylthiazoyl-2-yl)-2,5-diphenyltetrazolium bromide (MTT) to a blue formazan which can be read spectrophotometrically. Cells, such as human H460 cells grown in 25 cm² flasks are passaged (passage number <20), resuspended in fresh RPMI cell culture medium and added into 96-well cell culture plates at a concentration of 1000 cells per well in 100 μL per well. The cells are then allowed to incubate for 24 hours at 37° C., 5% CO₂. The following day, drug combinations at the specified ratios are prepared in 500 μL centrifuge tubes. The fixed ratio combinations are then serially diluted using liquid handling robots with fresh RPMI cell culture media and are administered to the appropriate or specified wells for single agents (100 μL) and at specific fixed ratio dual agent combinations (increments of 100 μL). The total well volumes should now be 200 μL. The drug exposure is for a duration of 72 hours.

Following drug exposure, MTT reagent (1 mg/mL phosphate buffered salt solution) is added to each well at a volume of 50 μL per well and incubated for 4 hours. The well contents are then aspirated and 150 μL of dimethylsulfoxide (DMSO) is added to each well to disrupt the cells and to solubilize the formazan precipitate within the cells. The 96-well plates are shaken on a plate shaker for a minimum of 2 minutes, and read on a microplate spectrophotometer set at a wavelength of 570 nm. The optical density (OD) readings are recorded and the OD values of the blank wells containing media alone are subtracted from all the wells containing cells. The cell survival following exposure to agents is based as a percentage of the control wells cells not exposed to drug. All wells are performed in triplicate and mean values are calculated.

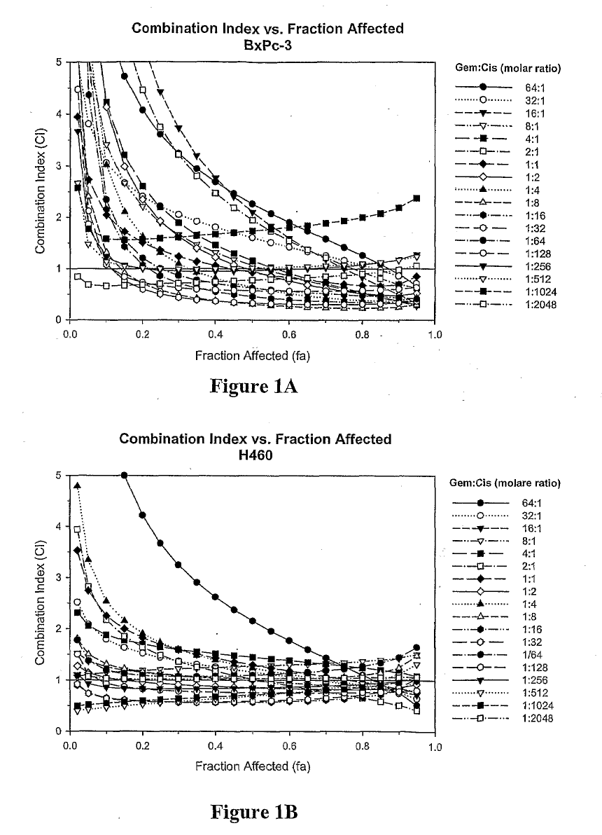

A combination index is then determined for each gemcitabine:cisplatin dose using CalcuSyn which is based on Chou and Talalay's theory of dose-effect analysis, in which a “median-effect equation” has been used to calculate a number of biochemical equations that are extensively used in the art. Derivations of this equation have given rise to higher order equations such as those used to calculate Combination Index (CI). As mentioned previously, CI can be used to determine if combinations of more than one drug and various ratios of each combination are antagonistic (CI>1.1), additive (0.9 CI 1.1) or synergistic (CI<0.9). CI plots are typically illustrated with CI representing the y-axis versus the proportion of cells affected, or fraction affected (fa), on the x-axis. The data in FIGS. 1A and 1B, plotted as CI versus the fraction of BxPc-3 or H460 human cancer cells affected (fa), respectively, illustrates that particular combinations of gemcitabine and cisplatin are antagonistic while others are particularly synergistic or additive.

Table 1 is a summary of CI values for screening data obtained from 6 cell lines.

TABLE 1
Combination Index values for Gem:Cis in 6 Cell Lines

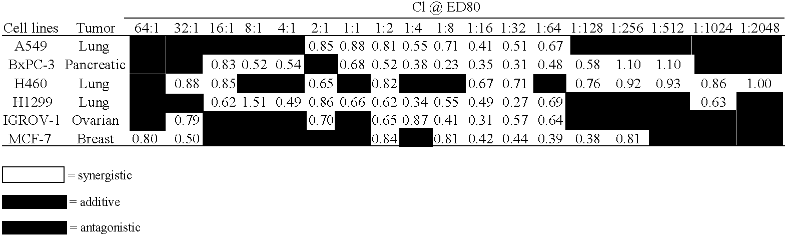

### Example 2

### Maintaining Ratios of Gemcitabine and Cisplatin In Vivo When Co-Encapsulated in the Same Liposome

To determine if liposome-loaded gemcitabine and cisplatin could be maintained at a fixed drug:drug ratio in the synergistic range in vivo, DSPC:DSPG:Chol (7:2:1; mol:mol), liposomes containing co-encapsulated gemcitabine and cisplatin at predetermined ratios were administered intravenously to mice and the plasma drug:lipid and drug:drug ratios were monitored over time.

Liposomes containing both gemcitabine and cisplatin could be generated using DSPC:DSPG:Cholesterol (7:2:1 mole ratio) liposomes containing passively entrapped cisplatin and subsequently loading them with gemcitabine. Briefly, lipid foams were prepared by dissolving lipids (DSPC:DSPG:CHOL (7:2:1 mol ratio)) mixed at a concentration of 100 mg lipid/ml final concentration into a chloroform:methanol:H₂O mixture (95:4:1; vol/vol). The solvent was then removed by vacuum evaporation and the resulting lipid foams were hydrated with a solution consisting of 150 mM saline, 8.5 mg/ml cisplatin at 75° C. The resulting MLVs were extruded at 75° C. to generate large unilamellar vesicles. The mean diameter of the resulting liposomes was determined by QELS (quasi-elastic light scattering) analysis to be approximately 100 nm+/−20 nm. Subsequently, the unencapsulated cisplatin was removed using a tangential flow column equilibrated in 150 mM saline.

Gemcitabine in 150 mM saline was added to these liposomes such that the final gemcitabine to cisplatin molar ratio would be about 1:1, 3:1, 6:1 or 10:1. Gemcitabine loading into the liposomes was facilitated by incubating the samples at 60° C. for 60 minutes. After loading, the sample was cooled to room temperature and unencapsulated gemcitabine was removed using tangential flow dialysis in 150 mM saline. Gemcitabine to lipid ratios were determined using liquid scintillation counting to determine lipid concentrations (¹⁴C-DPPC) and gemcitabine concentrations (³H-gemcitabine). Cisplatin loading efficiency was measured using Atomic Absorbance against a standard curve.

The preparation was then injected intravenously via the tail vein into CD-1 nude mice. Doses of the liposomal formulations for gemcitabine:cisplatin ratios of 1:1, 3:1, 6:1 and 10:1 were 3.8, 2.3, 2.5 and 1.0 mg/kg of cisplatin, respectively and 3.7, 6.4, 15.8 and 12.1 mg/kg of gemcitabine, respectively. At the indicated time points after intravenous administration, blood was collected by cardiac puncture (3 mice per time point) and placed into EDTA coated micro containers. The samples were centrifuged to separate plasma, and plasma was transferred to another tube. Gemcitabine and cisplatin plasma levels were quantified with High Performance Liquid Chromatography (HPLC).

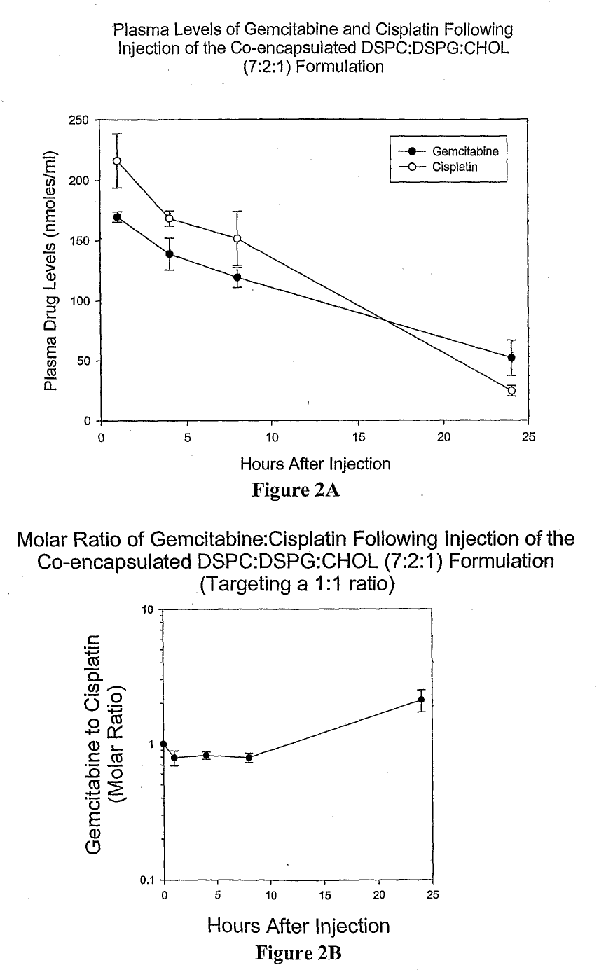

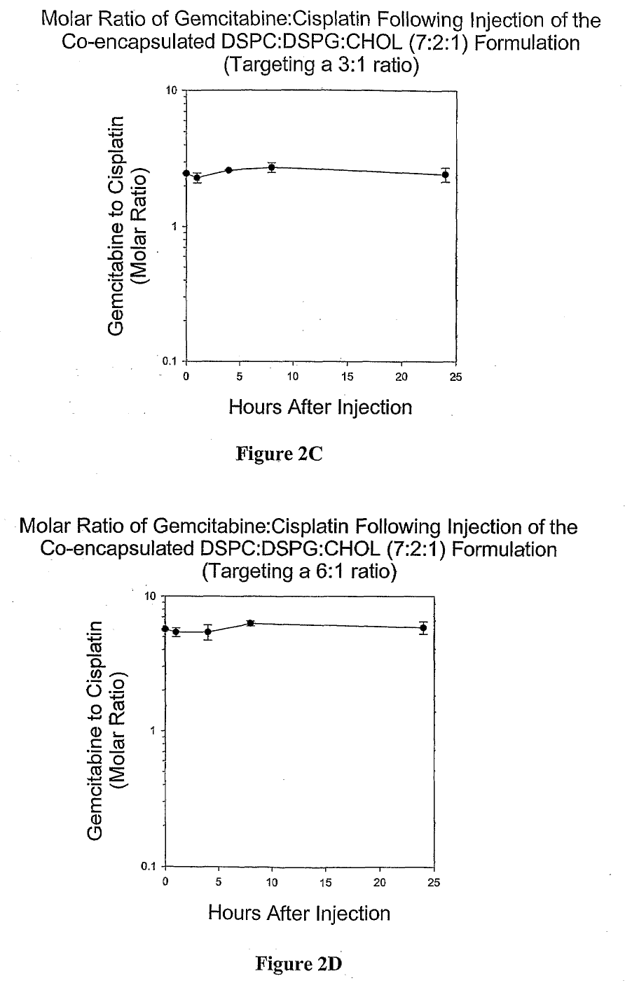

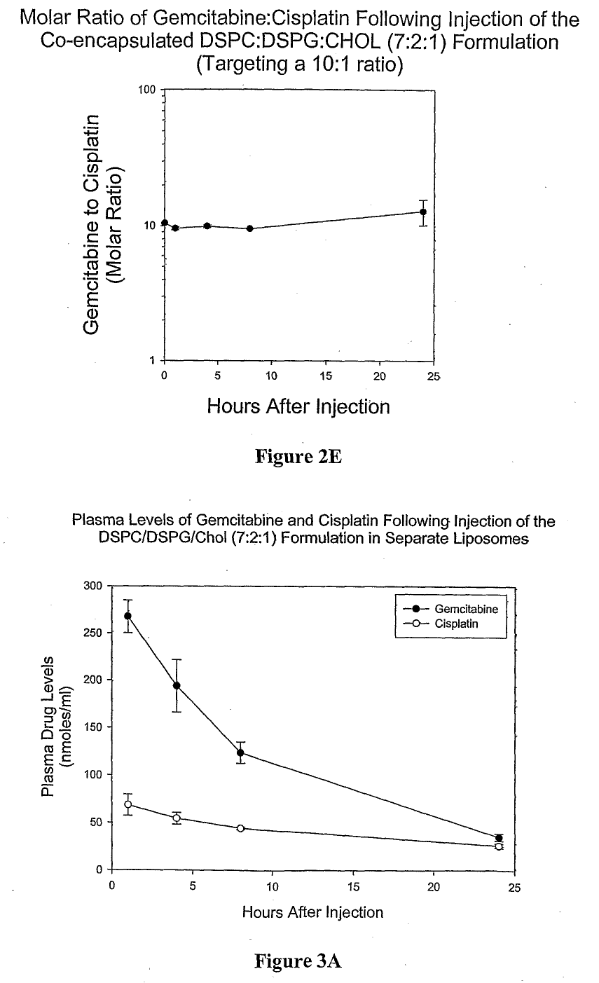

FIG. 2A shows the plasma drug concentration of gemcitabine and cisplatin at various time points after intravenous administration to CD-1 mice when they were delivered in the above-described liposomes at the 1:1 molar ratio.

FIGS. 2B, 2C, 2D and 2E respectively show that plasma levels of gemcitabine and cisplatin were effectively maintained at about a 1:1, 3:1, 6:1 or 10:1 drug ratio for an extended time after intravenous administration to CD-1 mice when the drugs were simultaneously delivered in the above-described liposomes. Data points represent the molar ratios of gemcitabine:cisplatin determined in plasma (+/−standard deviation) at the specified time points. Therefore, appropriately designed delivery vehicles such as liposomes can deliver desired ratios of gemcitabine and cisplatin in vivo.

### Example 3

### Maintaining Ratios of Gemcitabine and Cisplatin In Vivo in Separate Liposomes

Pharmacokinetics studies were carried out to determine if the liposome loaded gemcitabine and cisplatin could be maintained at a fixed drug:drug ratio where the drug was encapsulated separately in liposomes of the same lipid composition. The same methods of production as for the co-encapsulated gemcitabine and cisplatin-containing liposomes were used, except in two respects. The first difference was that the gemcitabine was loaded under the same conditions into empty liposomes. The second difference was that the cisplatin loading protocol was modified for this study. Liposomes extruded in 150 mM NaCl were added to a solution of 8 mg/ml cisplatin at 60° C. Ethanol was then added dropwise until a final ethanol concentration of 8% by volume was achieved. Cisplatin uptake was allowed to occur at 60° C. for 60 minutes, at which time the liposomes were cooled to 4° C. to allow cisplatin to precipitate. Precipitated cisplatin was removed by centrifugation at 2000 rpm for two minutes. Any further unencapsulated cisplatin was removed using a tangential flow column.

The liposomal gemcitabine and the liposomal cisplatin were combined in the same solution, and then injected intravenously via the tail vein into B6D2F1/Hsd mice. Doses of the liposomal formulation were 5.0 mg/kg of gemcitabine and 1.7 mg/kg cisplatin for the DSPC:DSPG:CHOL (7:2:1) formulation and 5.0 mg/kg gemcitabine and 2.0 mg/kg cisplatin for the DSPC:DPPC:DSPG:CHOL (35:35:20:10) formulation. At the indicated time points after intravenous administration, blood was collected by cardiac puncture (3 mice per time point) and placed into EDTA coated micro containers. The samples were centrifuged to separate plasma, and plasma was transferred to another tube. Gemcitabine and cisplatin plasma levels were quantified with High Performance Liquid Chromatography (HPLC). Data points represent the molar concentrations and molar ratios of gemcitabine:cisplatin determined in plasma (+/−standard deviation) at the specified time points.

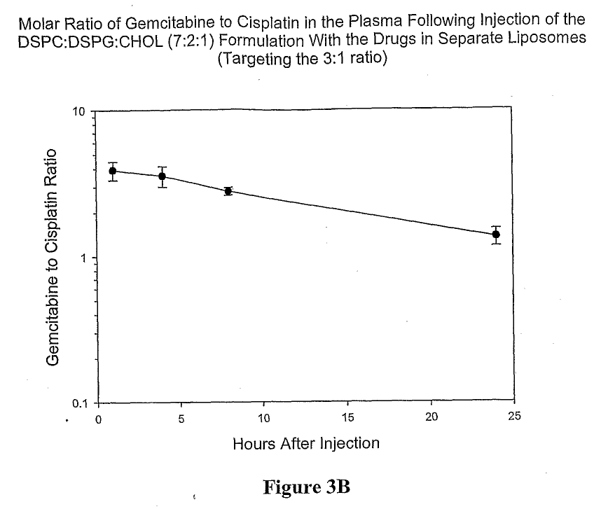

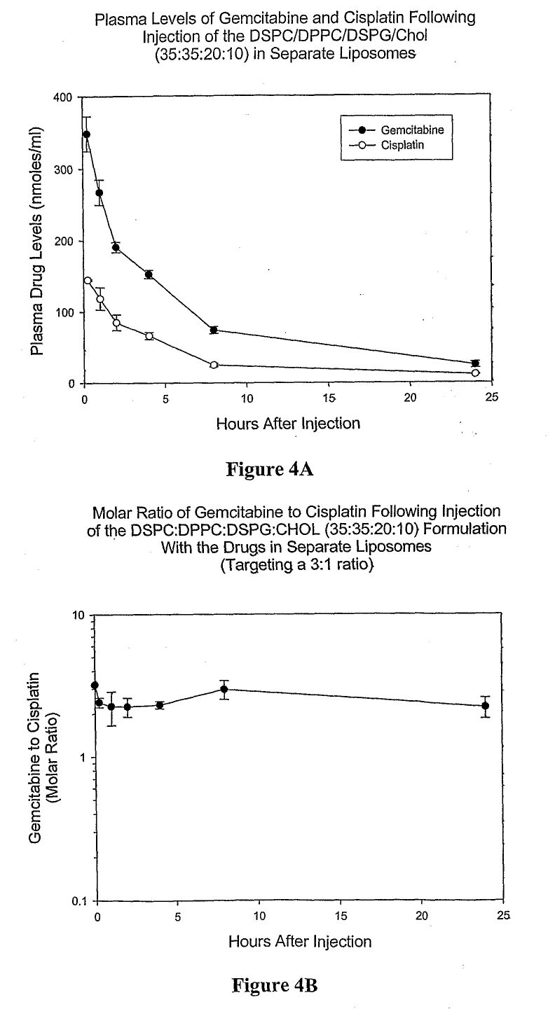

FIGS. 3A, 3B, 4A, and 4B show that plasma levels of gemcitabine and cisplatin were effectively maintained at about a 3:1 gemcibatine:cisplatin ratio for an extended time after intravenous administration to B6D2F1/Hsd mice when the drugs were delivered in separate liposomes composed of both DSPC:DSPG:CHOL (7:2:1) and DSPC:DPPC:DSPG:CHOL (35:35:20:10). Comparable PK results were achieved in a study in which the DSPG was replaced by DPPG in the preparation of separately encapsulated gemcitabine and cisplatin liposomes while keeping all other preparation methods constant, such that the formulation was DSPC:DPPC:DPPG:CHOL (35:35:20:10) (data not shown). Therefore, multiple appropriately designed delivery vehicles can deliver desired ratios of gemcitabine and cisplatin in vivo when the drugs are formulated in separate liposomes of the same composition.

### Example 4

### Efficacy of Gemcitabine and Cisplatin Containing Liposomes at a 3:1 Molar Ratio Against the Murine P388 Leukemia Ascites Tumor

To determine the anti-cancer efficacy of single agent and fixed ratio (3:1 gemcitabine:cisplatin molar ratio) liposomal gemcitabine and cisplatin, DSPG:DSPG:Chol (7:2:1; mol:mol) separately loaded gemcitabine and cisplatin-containing liposomes were administered intravenously to mice with P388 ascites tumors and their survival was evaluated. A gemcitabine to cisplatin molar ratio of 3:1 was selected because it is non-antagonistic in the majority of cell lines tested while providing the greatest drug dose intensity for each agent.

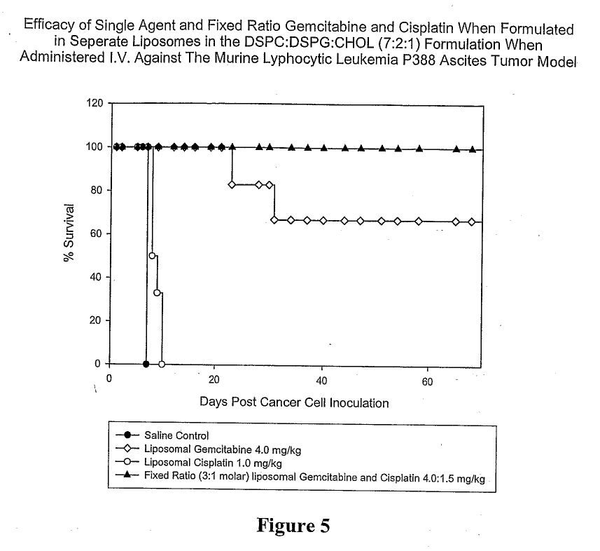

P388 cells from the NCI tumor repository were maintained as an ascitic fluid in the BDF-1 mouse, passaged to new mice weekly. Mice were euthanized, and the ascitic cells aseptically removed through the abdominal wall with a 20 g needle. For tumor implantation, cells were rinsed with Hanks Balanced Salt Solution (HBSS), counted on a heamocytometer and diluted with HBSS to a concentration of 2×10⁶ cells/ml. Mice were randomly grouped (n=6/group) and inoculated (Day=0) with 1×10⁶ cells implanted intraperitoneally in the BDF-1 mouse in a volume of 500 μL with a 25 g needle. Mice were injected with the required volume to administer the prescribed dose (mg/kg) to the animals based on individual mouse weights where the injection volume was 200 μL/20 g mouse for i.v. injections of drug. Three individual doses of 4.0 mg/kg (15 μlμmol/kg) [12.0 mg/kg; 45 μmol/kg total] liposomal gemcitabine and 1.0 mg/kg (3.3 μmol/kg) [3.0 mg/kg; 10 μmol/kg total] liposomal cisplatin on a Q4D schedule starting on Day 1 were compared with two individual doses of the fixed ratio combination using 4.0 mg/kg (15 μmol/kg) liposomal gemcitabine and 1.5 mg/kg (5 μmol/kg) cisplatin [3.0 mg/kg; 10 μmol/kg total cisplatin and 8.0 mg/kg; 30 μmol/kg total gemcitabine]. Ascitic tumor progression and survival were monitored twice daily Monday through Friday and once on the weekend in order to determine efficacy as a function of increased life span. Survival rates for animals needing to be terminated due to tumor progression were logged as the following day. Results are presented in FIG. 5.

Liposomes containing gemcitabine resulted in four long-term survivors of >70 days. Liposomes containing cisplatin resulted in only a one day increase in median survival time. However, the combination of liposomal cisplatin and liposomal gemcitabine resulted in 100% long-term survivors of >70 days even though liposomal cisplatin alone had negligible efficacy as well as the fact that only two doses of the combination were used containing a lower total gemcitabine [8.0 mg/kg; 30 μmol/kg compared to 12.0 mg/kg; 45 μmol/kg] and an equivalent-total cisplatin as the individual cisplatin containing liposomes [3.0 mg/kg; 10 μmol/kg].

### Example 5

### Efficacy of Low-Dose Gemcitabine and Cisplatin-Containing Liposomes at a 3:1 Ratio Demonstrates In Vivo Synergy Against the Murine P388 Leukemia Ascites Tumor Model

To determine synergy of the anti-cancer efficacy of single agent and fixed ratio liposomal gemcitabine and cisplatin (3:1 drug:drug molar ratio), DSPC:DPPC:DPPG:Chol (35:35:20:10) gemcitabine and cisplatin-loaded liposomes were administered at low doses (approximately 50% MTD) intravenously to mice with P388 ascites tumors, and their survival was evaluated as increased life span and log cell kill.

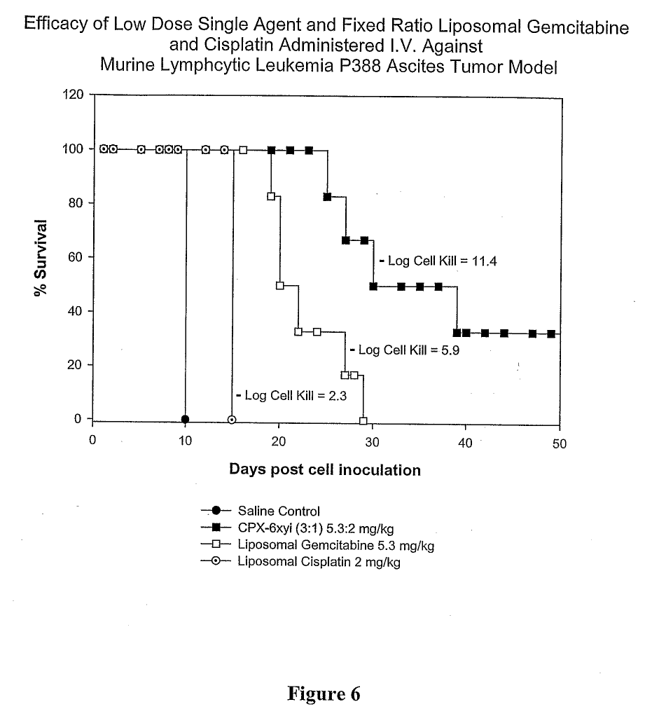

P388 cells from the NCI tumor repository were maintained as an ascitic fluid in the BDF-1 mouse, passaged to new mice weekly. Mice were euthanized, and the ascitic cells aseptically removed through the abdominal wall with a 20 g needle. For tumor implantation, cells were rinsed with Hanks Balanced Salt Solution (HBSS), counted on a heamocytometer and diluted with HBSS to a concentration of 2×10⁶ cells/ml. Mice were randomly grouped (n=6/group) and inoculated (Day=0) with 1×10⁶ cells implanted intraperitoneally in the BDF-1 mouse in a volume of 500 μL with a 25 g needle. Mice were injected with the required volume to administer the prescribed dose (mg/kg) to the animals based on individual mouse weights where the injection volume was 200 μL/20 g mouse for i.v. injections of drug. Three individual doses of 5.3 mg/kg (20 μmol/kg) liposomal gemcitabine and 2.0 mg/kg (6.7 mmol/kg) liposomal cisplatin on a Q4D schedule starting on Day 1 were compared with three individual doses of the fixed ratio combination using 5.3 mg/kg (20 μmol/kg) liposomal gemcitabine and 2.0 mg/kg (6.7 μmol/kg) cisplatin. Ascitic tumor progression and survival were monitored twice daily Monday through Friday and once on the weekend in order to determine efficacy as a function of increased life span. Survival rates for animals needing to be terminated due to tumor progression were logged as the following day. Tumor growth delay was calculated based on the median increased life span. The tumor cell doubling rate for the P388 cells was determined to be 16 hours based on previous cell titration experiments. Cell kill was calculated from the survival curves using the formula: log₁₀ cell kill=tumor growth delay/3.32×T_{d}, in which 3.32 is the number of cell doublings per log of growth and T_{d} is the tumor doubling time (Corbett and Valeriote, 1987 Rodent models in experimental chemotherapy. In Rodent Tumor Models in Experimental Cancer Therapy, Kallman R F (ed.), pp 233-247, Pergamon Press: New York). Results are shown in FIG. 6.

Liposomes containing gemcitabine resulted in a log cell kill of 5.9. Liposomes containing cisplatin resulted in a log cell kill of 2.3. Additive anti-tumor activity would predict a log cell kill value of 8.2 for the combination. However, the combination of liposomal cisplatin and liposomal gemcitabine at the same respective doses resulted in a log cell kill of 11.4, clearly demonstrating a more than additive increase in efficacy when the two agents are combined in liposomes at the 3:1 gemcitabine:cisplatin ratio and the ratio is maintained in the blood after administration.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

1. A composition comprising delivery vehicles, which are particles of size dependent on the route of administration said delivery vehicles having stably associated therewith at least one platinum agent and one cytidine analog at a cytidine analog-to-platinum agent mole ratio that is non-antagonistic in vitro or in vivo and wherein a non-antagonistic ratio is maintained in the blood after administration.

2. The composition of claim 1, wherein the platinum agent is selected from the group consisting of cisplatin, carboplatin and oxaliplatin.

3. The composition of claim 1, wherein the cytidine analog is selected from the group consisting of cytarabine, gemcitabine and 5-azacytidine.

4. The composition of claim 2, wherein the cytidine analog is selected from the group consisting of cytarabine, gemcitabine and 5-azacytidine.

5. The composition of claim 1, wherein the cytidine analog is gemcitabine and the platinum agent is cisplatin.

6. The composition of claim 1, wherein the cytidine analog and platinum agent are co-encapsulated.

7. The composition of claim 1, wherein the cytidine analog and platinum agent are encapsulated in separate delivery vehicles.

8. The composition of claim 1, wherein the composition comprises a third agent.

9. The composition of claim 1, wherein the delivery vehicles have a mean diameter of between 4.5 and 500 nm.

10. The composition of claim 9, wherein the delivery vehicles have a mean diameter of less than 300 nm.

11. The composition of claim 1, wherein the delivery vehicles comprise liposomes, lipid micelles, block copolymer micelles, microparticles, nanoparticles, and/or derivatized single chain polymers.

12. The composition of claim 11, wherein the delivery vehicles comprise liposomes.

13. The composition of claim 12, wherein the liposomes comprise phosphatidylcholine-containing lipids, and/or
wherein the liposomes comprise a charged lipid; and/or
wherein the liposomes comprise sphingomyelin; and/or
wherein the liposomes comprise a sterol.

14. The composition of claim 13, wherein the phosphatidylcholine-containing lipid is DPPC, DSPC or DAPC; and/or
wherein the charged lipid is DSPG, DPPG or DMPG or a phosphatidyl inositol and/or
wherein the sterol is cholesterol.

15. A method to treat a disease condition in a subject which method comprises administering to a subject in need of such treatment a therapeutically effective amount of the composition of claim 1.

16. A method to deliver a therapeutically effective amount of a cytidine analog:platinum agent combination by administering a cytidine analog stably associated with a first delivery vehicle and a platinum agent stably associated with a second delivery vehicle, wherein the mole ratio of the cytidine analog and the platinum agent delivered is non-antagonistic in vitro and in vivo and wherein the non-antagonistic ratio is maintained in the blood after administration.
